(19) United States
(12) Patent Application Publication
(10) Pub. No.: US 2007/0238785 A1
(43) Pub. Date: Oct. 11, 2007

(54) Use of an anti-atherothrombotic compound in obtaining medicaments intended for the treatment of vascular disorders

(75) Inventors: Marie-Dominique Fratacci, Bois D'arcy (FR); Agnes De Cordoue, Paris (FR); Laurence Lerond, Marly Le Roi (FR); Tony Verbeuren, Vernouillet (FR); Alain Rupin, Savonnieres (FR); Gilbert Lavielle, La Celle Saint Cloud (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE cedex (FR)

(21) Appl. No.: 11/784,070

(22) Filed: Apr. 5, 2007

(30) Foreign Application Priority Data
Apr. 7, 2006 (FR) 06/03082

(51) Int. Cl. A61K 31/195

(52) U.S. Cl. 514/562

(57) ABSTRACT

The present invention relates to the use of a specific antagonist of TP receptors in obtaining medicaments intended for the treatment of vascular disorders in patients having had a previous cerebral vascular accident, especially for reducing cerebrovascular and cardiovascular events in patients having had a previous cerebral ischaemic accident (CIA) or transitory ischaemic attack (TIA).

The present invention relates to the use of a specific antagonist of thromboxane-prostaglandin (TP) receptors in obtaining medicaments intended for the treatment of vascular disorders in patients having had a previous cerebrovascular accident (CVA), especially for reducing cerebrovascular and cardiovascular events in patients having had a previous cerebral ischaemic accident (CIA) or transitory ischaemic attack (TIA).

Thromboxane A₂ (TXA₂) is an unstable metabolite of arachidonic acid which is involved in the pathogenesis of numerous cardiovascular disorders.

TXA₂ and other metabolites of arachidonic acid such as endoperoxides (PGG₂/PGH₂), HETEs and isoprostanes are ligands of common receptors called TP receptors (thromboxane—prostaglandins—endoperoxides). Their binding to TP receptors brings about harmful effects: platelet activation and aggregation, endothelial dysfunction, vasoconstriction and cellular proliferation.

Atherosclerosis is a chronic inflammatory disease, the injurious agent that causes the inflammatory reaction being LDL cholesterol which accumulates under the endothelium in oxidised form. Inflammation is involved at several levels in the atheromatous process: 1) activation of the endothelium and monolymphocyte recruitment; 2) local and systemic production of pro-inflammatory cytokines; 3) production of proteases of the extracellular matrix (metalloproteases) causing degradation of the proteins of the fibrous cap and destabilisation of the plaque; 4) induction of apoptosis of the cells of the plaque and formation of a pro-coagulant lipid core.

The atheromatous plaque thereby formed can rupture as a result of stimuli caused by local inflammation and oxidative stress, initiating the aggregation of platelets on the surface which result in occlusion. The occurrence of thrombosis on plaque is called “atherothrombosis”.

Rupture involves mainly (but not exclusively) plaques which have a large lipid core, occupying more than 40% of the total volume of the plaque, and a fine fibrous cap which is rich in macrophages and poor in smooth muscle cells. It is the result of the disequilibrium between the haemodynamic stresses to which the fibrous cap is subjected and the intrinsic solidity which governs its resistance to fracture.

In recent therapeutic strategies for atherosclerosis it appears to be important to target the inflammation in the plaque. Stabilisation of the atheromatous plaque necessitates reduction of its inflammatory component in favour of its fibrous component.

Recently, a large number of research studies have been carried out with the aim of preventing the phenomena associated with the excessive production of thromboxane A₂ in the cardiovascular and neurovascular systems. Among such antagonists, those described in the patent specification EP 648 741 have proved to be powerful and selective antagonists of TP receptors, to be active by the oral route and to have a long duration of action.

### BRIEF DESCRIPTION OF THE FIGURES

FIG. 1: Representation of the percentage survival of treated and untreated sodium-loaded SHR-SP rats over time.

FIG. 2: Representation of cardiac hypertrophy as a percentage of body weight in treated and untreated sodium-loaded SHR-SP rats.

FIG. 3: Representation of oxidative stress as a function of 8-OH-dG present in urine (ng/24 hours) in treated and untreated sodium-loaded SHR-SP rats.

FIG. 4: Representation of acute inflammation as a function of plasma thiostatin (μg/ml) in treated and untreated sodium-loaded SHR-SP rats.

More specifically, 3-{6-[(4-chlorophenylsulphonyl)amino]-2-methyl-5,6,7,8-tetrahydro-naphth-1-yl}propionic acid of formula (I): 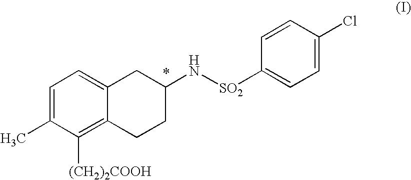
in racemic form or in the form of an optically pure isomer and also pharmaceutically acceptable salts thereof, is a specific antagonist of TP receptors, which has been found to have a powerful anti-atherothrombotic effect.

The compound of formula (I) acts by blocking platelet aggregation induced by thromboxane A₂ and the other TP receptor ligands, whatever their origin (platelet or extra-platelet). It further acts by inhibiting the vasoconstriction induced by thromboxane A₂ and by opposing endothelial dysfunction, proliferation and also inflammation of the vascular wall.

The compound of formula (I) accordingly has anti-platelet aggregation, anti-thrombotic, anti-inflammatory, anti-proliferative and anti-vasoconstrictive properties.

Its beneficial effect in atherosclerosis may be a reflection of the inhibition of monocyte/macrophage infiltration into the plaque, probably by way of the inhibition of the expression of vascular adhesion molecules. Because such inflammatory cells affect the stability of the plaque, the reduction in monocyte recruitment into the vascular wall may, in the long term, stabilise the plaque. In addition, the anti-thrombotic effect of the compound may reduce the vascular inflammation and endothelial activation governed by adhesion of platelets to the endothelium and the pro-coagulating and pro-oxidative interaction, which is inflammatory in nature, between platelets, endothelium, smooth muscle cells and monocytes.

3-{6-[(4-Chlorophenylsulphonyl)amino]-2-methyl-5,6,7,8-tetrahydronaphth-1-yl}-propionic acid, its salts and its optical isomers, preparations thereof and the use thereof in therapeutics have been described in the European Patent specification EP 0 648 741.

The Applicant has now discovered, surprisingly, that the compound of formula (I) in the form of an active isomer and in salt form, preferably of configuration (R) and in sodium salt form, has valuable properties allowing it to be used in the treatment of vascular disorders in patients having had a previous cerebrovascular accident, especially for reducing cerebrovascular and cardiovascular events in patients having had a previous CIA or TIA.

Surprisingly, the compound of formula (I) of configuration (R) preferably in sodium salt form does not behave like other anti-platelet aggregation agents: indeed, at low doses, aspirin does not have a direct anti-atherosclerotic or anti-atherogenic effect. It does not inhibit neointima formation and the accumulation of inflammatory cells in the vascular wall. Vascular effects in humans have not been claimed for clopidogrel either. P2Y12 receptors, which are the target of clopidogrel, are not present in endothelial cells.

We have been able to demonstrate, surprisingly, these secondary protection properties of the compound of formula (I) in an animal model: the sodium-loaded SHR-SP rat. These properties seem to be connected, surprisingly, to the compound's causing a reduction in oxidative stress. The importance of oxidative stress in cerebral pathology has been demonstrated in several publications (Margaill I, Plotkine M, Lerouet D, Free Radical Biology & Medicine, 2005, 39, 429-443; Crack P J, Taylor J M, Free Radical Biology & Medicine, 2005, 38, 1433-1444). The discovery of an effect of the compound of formula (I) on oxidative stress within the context of cerebral vascular pathology accordingly suggests its possible use in a study such as that described in the present invention.

SHR-SP rats represent a well-characterised experimental model of essential hypertension. These rats develop severe spontaneous hypertension leading rapidly to the formation of cerebral lesions. They are highly sensitive to a salt-enriched diet, which causes the development of malignant nephrosclerosis (Griffin K A, Churchill P C, Picken, Webb R C, Kurtz T W, Bidani A K, Am. J. Hypertens., 2001, 14(4 Pt 1): 311-20), leads to an increase in arterial pressure and in oxidative stress (Park J B, Touyz R M, Chen X, Schiffrin E L, Am. J. Hypertens., 2002, 15(1 Pt1): 78-84; Manning R D, Meng S, Tien N, Acta Physiol. Scand, 2003, 179:243-250) and aggravates vascular lesions and cardiac hypertrophy (Kyselovic J, Morel N, Wibo M, Godfraind T, J. Hypertens., 1998, 16(10): 1515-22). Furthermore, SHR-SP rats are the only animal model which spontaneously develops complex cerebrovascular disorders (Volpe M, Russo R, Veccione C, Savoia C, Piras O, Gigante B, Lindpaintner K, Rubattu S, J. Hypertens., 1998, 16: S31-S35; Guerrini U, Sironi L, Tremoli E, Cimino M, Pollo B, Calvio A M, Paoletti R, Asdente M, Stroke, 2002, 33(3): 825-30). Cerebral analysis of SHR-SP rats by ESR (electron spin resonance) spectroscopy shows an elevated level of oxidative stress compared to normotensive WKY rats (Miyazaki H, Shoji H, Lee M C, Redox Rep., 2002, 260-2657). It has been suggested that this increase in oxidative stress might bring about disruption of the blood-brain barrier (Kim-Mitsuyama S, Yamamoto E, Tanaka T, Zhan Y, Izumi Y, Izumiya Y, Ioroi T, Wanibuchi H, Iwao H, Stroke, 2005, 36: 1077-1082) as a consequence of lesions of cellular lipid membranes caused by peroxidation and by DNA and protein damage. These cerebral disorders are often associated with clinical behavioural signs (lethargy or aggressiveness, convulsions, limb paralysis) preceding the occurrence of cerebral vascular accidents resulting in the death of the animals.

The sodium-loaded SHR-SP rat model appears to be a good target for evaluation of the compound of formula (I) in the prevention of acute cerebral accident. Indeed, selecting a period of treatment of between 10 and 15 weeks provides a perfect model of recurrent episodes of cerebral attack. Prior to the 10 weeks, the rats have already undergone non-fatal cerebral attacks (e.g. lethargy, convulsions) and as a result we can analyse whether or not the compound of formula (I) is capable of preventing or delaying secondary cerebral accidents.

In this respect, this rat model resembles the human situation and, more specifically, the situation of the study described in the patent application specification, in which the patients have had a first cerebral accident and which study is directed at the effect of treatment with the compound of formula (I) on prevention of a second event (cerebral or cardiovascular), which has the risk of being fatal.

The invention accordingly relates to use of the compound of formula (I) in the form of an active isomer and preferably in salt form in obtaining pharmaceutical compositions intended for the treatment of vascular disorders in patients having had a previous cerebrovascular accident, especially for reducing cerebrovascular and cardiovascular events in patients having had a previous CIA or TIA.

Among the addition salts of the compound of formula (I) there may be mentioned, without implying any limitation, addition salts with a pharmaceutically acceptable base such as the salts of sodium, potassium, tert-butylamine, diethylamine etc.

Preferably, the salt used will be the sodium salt.

In the compositions according to the invention, the compound of formula (I) preferably has the absolute configuration (R).

The pharmaceutical compositions will be provided in forms suitable for administration by the oral, parenteral, transcutaneous, nasal, rectal and perlingual route, especially in the form of injectable preparations, tablets, sublingual tablets, glossettes, gelatin capsules, capsules, lozenges, suppositories, creams, ointments, dermal gels etc.

The pharmaceutical compositions according to the invention comprise one or more excipients or vehicles selected from diluents, lubricants, binders, disintegrating agents, absorbents, colourants, sweeteners etc.

The dosage used does not vary according to the sex, age or weight of the patient, the nature of the disorder or any associated treatments.

The studies were carried out using the sodium salt of the (R) isomer of the compound of formula (I).

In the compositions according to the invention, the amounts of active ingredient are from 5 to 100 mg for the sodium salt of the (R) isomer of the compound of formula (I). Preferably, the daily dose of the sodium salt of the (R) isomer of the compound of formula (I) will be 30 mg per day.

Preclinical Data

The anti-thrombotic effect of the sodium salt of the (R) isomer of the compound of formula (I) was demonstrated in various models of carotid thrombosis in vivo: vascular electrolysis (dog), mechanical lesion (guinea pig) and electric stimulation (rat), and ex vivo: carotid-carotid arterial bypass by means of a glass capillary: the sodium salt of the (R) isomer of the compound of formula (I) lengthens the occlusion time in dose-dependent manner.

The anti-proliferative, anti-atheromatous and anti-inflammatory effects of the sodium salt of the (R) isomer of the compound of formula (I) were explored in experimental models: it significantly reduces the surface area of the atherosclerotic lesion, the thickness of the vascular wall and the intima/media ratio without reducing serum cholesterol. It also inhibits the infiltration of macrophages into the vascular wall and the expression of adhesion molecules and especially markers such as endothelin, ICAM and VCAM.

Animal Model: Sodium-Loaded SHR-SP Rat

Materials and Methods

Monitoring of Animals

10-week old male SHR-SP rats (Charles River) being given 1% NaCl in their drinking water were separated into two groups in random manner and were treated, or not, with the sodium salt of the (R) isomer of the compound of formula (I) (30 mg/kg/day) incorporated in their food.

In a first study, the survival of each group was determined by calculating the percentage of animals alive each day after the start of treatment.

In a second study, the rats were monitored over 5 weeks, between 10 and 15 weeks of age, and were then sacrificed in order to quantify a urinary marker of oxidative stress (8-OH-dG) and a plasma marker reflecting acute inflammation (thiostatin, which in the rat is the equivalent of CRP) and in order to determine cardiac hypertrophy.

Physiological Parameters

During the last 24 hours of monitoring, the animals were placed in individual metabolic cages and the urines were collected in sterile tubes (200 μl of 2 mM EDTA). Diuresis was measured and the production of 8-hydroxy-2′-deoxyguanosine (8-OH-dG) was quantified by ELISA assay (Oxis, Bioxytech).

The rats were anaesthetised by intraperitoneal injection of pentobarbital sodium (50 mg/kg, Ceva laboratories). Blood was sampled from the carotid artery (sampling into heparin 5000 IU/ml) in order to measure thiostatin production by ELISA assay (Life Diagnostics). The heart was excised, dried and weighed, and cardiac hypertrophy was determined by dividing the weight of the dry heart by the body weight for each animal in order to standardise the measurement.

Statistics

The results are reported as the mean±S.E.M. Differences are considered significant when P<0.05 after a Student's t test. The survival of the animals is represented on a Kaplan-Meier graph and the results are considered significant when P<0.05 after a log-rank test.

Results

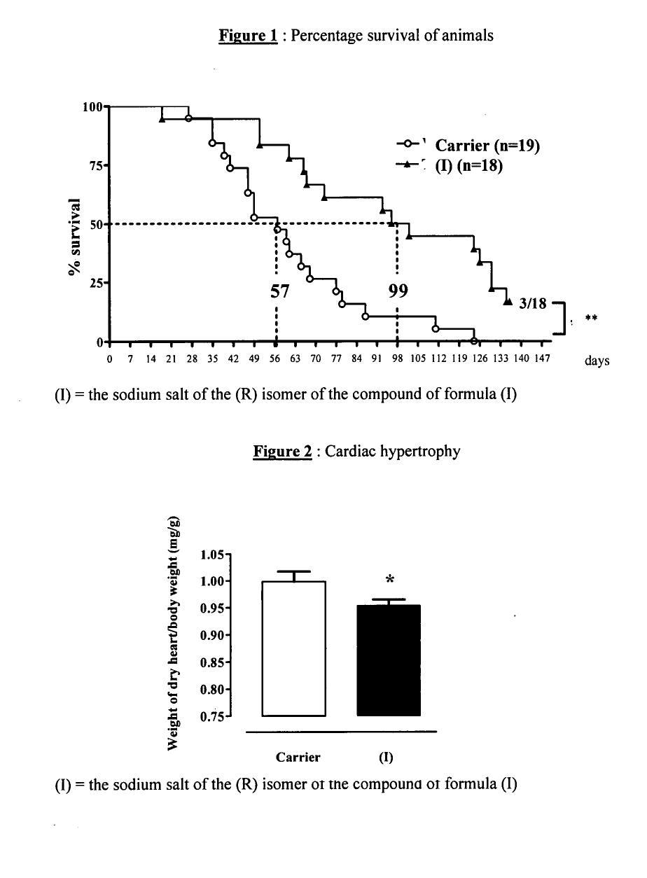

1) Percentage Survival of Animals (FIG. 1)

The percentage survival of animals was calculated for the two groups of rats. For the Carrier group, half the rats were dead 57 days after the start of monitoring whereas, in the group of rats treated with the sodium salt of the (R) isomer of the compound of formula (I), half the rats were dead after 99 days of treatment.

This result shows that the sodium salt of the (R) isomer of the compound of formula (I) makes it possible to prevent the animals from dying and therefore to improve the percentage survival.

2) Cardiac Hypertrophy (FIG. 2)

The rats in the Carrier group have a large increase in their cardiac mass, which reflects an increase in size, or hypertrophy, of that organ. When the animals are treated with the sodium salt of the (R) isomer of the compound of formula (I), hypertrophy of the heart is less marked.

This result shows that the sodium salt of the (R) isomer of the compound of formula (I) makes it possible to slow down cardiac hypertrophy.

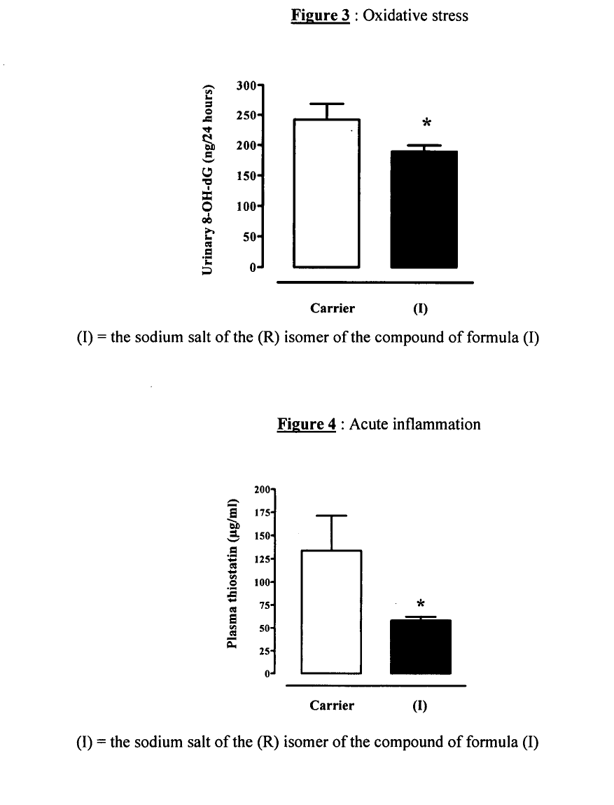

3) Oxidative Stress (FIG. 3)

The rats in the Carrier group exhibit oxidative stress, which can be determined by means of urinary markers such as 8-OH-dG. The total amount of 8-OH-dG present in the urines over 24 hours is reduced for the animals in the group receiving the sodium salt of the (R) isomer of the compound of formula (I) compared to the animals in the Carrier group.

This result shows that the sodium salt of the (R) isomer of the compound of formula (I) makes it possible to reduce the level of oxidative stress.

4) Acute Inflammation (FIG. 4)

Thiostatin is a protein produced during the acute phase of inflammation. This protein is markedly increased in the plasma of the rats in the Carrier group. When the animals are treated with the sodium salt of the (R) isomer of the compound of formula (I), the amount of plasma thiostatin is lower.

This result shows that the sodium salt of the (R) isomer of the compound of formula (I) makes it possible to slow down the establishment of acute inflammation.

Clinical Data

The ex vivo anti-thrombotic activity of the sodium salt of the (R) isomer of the compound of formula (I) was also assessed in patients at high risk of the occurrence of ischaemic CVA, and superiority of the compound of formula (I) over aspirin after 10 days of administration was demonstrated in respect of the main parameters of the perfusion chamber model (total surface area and dense surface area of the thrombus, % adhesion). In this study, the sodium salt of the (R) isomer of the compound of formula (I) also has an effect on the measured markers of inflammation (sVCAM, sPAI-1, sP-selectin, thrombomodulin).

Study of the peripheral vasomotor response on single or chronic administration of the compound of formula (I) to atheromatous patients having a high level of cardiovascular risk and an abnormal hyperaemia test and being treated with aspirin demonstrated superiority of the compound of formula (I) over placebo with regard to endothelium-dependent vasodilation evaluated by the provoked hyperaemia test (physiological stimulus of the radial artery). This superiority is observed whatever the dose, after single administration or repeated administration (15 days), the vasodilatory effect being evaluated about 2 hours after administration of the medicament.

### CONCLUSION

The compound of formula (I) in the form of the (R) isomer and preferably in the form of the sodium salt, which has anti-platelet aggregation and anti-thrombotic, anti-inflammatory, anti-proliferative and anti-vasoconstrictive properties, accordingly appears to be an anti-atherothrombotic agent for use in the secondary prevention of cardiovascular accidents in atherothrombotic diseases, especially in patients having had a previous cerebral ischaemic accident.

1. A method for treating or reducing a vascular disorder in a patient, wherein the patient has had a previous cerebral vascular accident, comprising administering to the patient, and amount of 3-{6-[(4-chlorophenylsulphonyl)amino]-2-methyl-5,6,7,8-tetrahydronaphth-1-yl}propionic acid of formula (I) in the form of an optical isomer or a pharmaceutically acceptable salt thereof which is effective for treatment of the vascular disorder.

2. The method of claim 1, wherein the vascular disorder is selected from cerebrovascular and cardiovascular events.

3. The method of claim 1, wherein the vascular disorder is selected from cerebrovascular and cardiovascular events and the previous cerebral vascular accident is a cerebral ischaemic accident.

4. The method of claim 1, wherein the vascular disorder is selected from cerebrovascular and cardiovascular events and the previous cerebral vascular accident is a transient ischaemic attack.

5. The method of claim 1, wherein the 3-{6-[(4-chlorophenylsulphonyl)amino]-2-methyl-5,6,7,8-tetrahydronaphth-1-yl}propionic acid of formula (I), has the (R) configuration and is in the form of a sodium salt.

6. A pharmaceutical composition comprising 3-{6-[(4-chlorophenylsulphonyl)amino]-2-methyl-5,6,7,8-tetrahydronaphth-1-yl}propionic acid of formula (I) in the form of an optical isomer or a pharmaceutically acceptable salt thereof, alone or in combination with one or more pharmaceutically acceptable exceipients.

7. The pharmaceutical composition of claim 6, wherein the 3-{6-[(4-chlorophenylsulphonyl)amino]-2-methyl-5,6,7,8-tetrahydronaphth-1-yl}propionic acid of formula (I) has the (R) configuration.

8. The pharmaceutical composition of claim 6, wherein the 3-{6-[(4-chlorophenylsulphonyl)amino]-2-methyl-5,6,7,8-tetrahydronaphth-1-yl}propionic acid of formula (I) of has the (R) configuration and is in the form of a sodium salt.

9. The pharmaceutical composition according to claim 6, wherein the amount of sodium salt of the (R) isomer of the compound of formula (I) is from 5 to 100 mg.

10. The pharmaceutical composition of claim 6, wherein the amount of sodium salt of the (R) isomer of the compound of formula (I) is 30 mg per day.
